(19) United States
(12) Patent Application Publication
(10) Pub. No.: US 2018/0303521 A1
(43) Pub. Date: Oct. 25, 2018

(54) SPINAL IMPLANT SYSTEM AND METHOD

(71) Applicant: Warsaw Orthopedic, Inc, Warsaw, IN (US)

(72) Inventor: Richard A. Hynes, Melbourne Beach, FL (US)

(21) Appl. No.: 15/492,843

(22) Filed: Apr. 20, 2017

(51) Int. Cl. A61B 17/70; A61F 2/44

(52) U.S. Cl. CPC A61B 17/7059; A61F 2002/30517; A61F 2/447

(57) ABSTRACT

A method for treating vertebrae includes the steps of: attaching a first spinal plate with cervical vertebrae to extend along a first intervertebral disc space; and attaching a second spinal plate with cervical vertebrae to extend along a second intervertebral disc space such that the plates are axially spaced apart a selected distance. Systems, instruments, spinal constructs and implants are disclosed.

### TECHNICAL FIELD

The present disclosure generally relates to medical devices for the treatment of musculoskeletal disorders, and more particularly to a surgical system and a method for treating a spine.

### BACKGROUND

Spinal pathologies and disorders such as degenerative disc disease, spondylolisthesis, disc herniation, osteoporosis, scoliosis and other curvature abnormalities, kyphosis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, corpectomy, laminectomy, fusion, fixation, correction and implantable prosthetics. As part of these surgical treatments, implants such as bone fasteners, interbody devices, plates, connectors and vertebral rods are often used to provide stability to a treated region. These implants can redirect stresses away from a damaged or defective region while healing takes place to restore proper alignment and generally support the vertebral members. This disclosure describes an improvement over these prior technologies.

### SUMMARY

In one embodiment, a method of treating vertebrae is provided. The method comprises the steps of: attaching a first spinal plate with cervical vertebrae to extend along a first intervertebral disc space; and attaching a second spinal plate with cervical vertebrae to extend along a second intervertebral disc space such that the plates are axially spaced apart a selected distance. In some embodiments, systems, instruments, spinal constructs and implants are disclosed.

### BRIEF DESCRIPTION OF THE DRAWINGS

The present disclosure will become more readily apparent from the specific description accompanied by the following drawings, in which:

FIG. 1 is a plan view of components of one embodiment of a system in accordance with the principles of the present disclosure;

FIG. 2 is a perspective view of components of one embodiment of a system in accordance with the principles of the present disclosure;

FIG. 3 is a perspective view of components of one embodiment of a system in accordance with the principles of the present disclosure disposed with vertebrae;

FIG. 4 is a perspective view of components of one embodiment of a system in accordance with the principles of the present disclosure disposed with vertebrae;

FIG. 5 is a perspective view of components of one embodiment of a system in accordance with the principles of the present disclosure disposed with vertebrae;

FIG. 6 is a plan view of components of one embodiment of a system in accordance with the principles of the present disclosure disposed with vertebrae;

FIG. 7 is a side view of the components and vertebrae shown in FIG. 6; and

FIG. 8 is a plan view of components of one embodiment of a system in accordance with the principles of the present disclosure disposed with vertebrae.

### DETAILED DESCRIPTION

The exemplary embodiments of a surgical system are discussed in terms of medical devices for the treatment of musculoskeletal disorders and more particularly, in terms of a spinal implant system for delivering and/or fastening implants with a surgical site and a method for treating a spine.

In some embodiments, the present spinal implant system includes a plurality of cervical plates employed with a method for treating a spine disorder. In some embodiments, the present spinal implant system includes one or more spinal constructs for treating multiple vertebral levels including a cervical plating system for treating a spine disorder. In some embodiments, the present spinal implant system includes a stacked cervical plating system employed with a method for treating a spine disorder in connection with an anterior cervical discectomy and fusion (ACDF).

In some embodiments, the present spinal implant system includes multiple single level cervical plates employed with a method of treating multi-level cervical disc degeneration. This configuration provides consistent and balanced forces on each disc level and attempts to avoid a lower rate of fusion due to imbalance of biomechanical forces on each disc level. In some embodiments, each of the multiple single level plates are placed with vertebrae and physically separated from the plate at the next level thereby allowing the plates to act independently. In some embodiments, the plates are spaced apart by a selected distance, such as, for example, a gap between the plates. In some embodiments, the gap is disposed in a cranial-caudal orientation relative to vertebrae. In some embodiments, the plates are spaced apart by a gap of at least 1 millimeter (mm). In some embodiments, the stacked cervical plating system includes a plurality of plates with a gap disposed between the plates from cephalad to caudal. In some embodiments, this configuration avoids overlapping between the plates. In some embodiments, the present plating system includes single level plates that are relatively offset laterally and/or alternating along vertebrae. In some embodiments, the plates are relatively offset laterally, for example, a distance in a range of 1-5 mm. In some embodiments, the offset orientation facilitates screw placement and/or avoids interference between adjacent screws. In some embodiments, the present plating system utilizes a multitude of single level plates that allow for more fixation screws per level. In some embodiments, the present plating system employs short plates such that bone screws can be placed in a hyper-angulated fashion for greater resistance to pull-out from vertebral tissue. In some embodiments, the present plating system utilizes multiple single level plates that allow for a global construct stiffness that is lower than that achieved with a single, elongated multi-level plate, for example, a single three vertebral level plate. As such, the present plating system provides for increased loading across bone grafts or cages placed between vertebrae and/or higher fusion potential. In some embodiments, the present plating system reduces incidence of pseudo arthrosis and/or prevents adjacent segment disease. In some embodiments, this configuration facilitates alignment of the plate screw holes with a vertebral body to optimize bone screw placement.

In one embodiment, the spinal implant system includes a spinal plate and a method of insertion thereof. In some embodiments, the present spinal implant system and method are employed with a surgical technique including a step of disc space distraction. In some embodiments, the method includes the step of performing discectomy/decompression under distraction. In some embodiments, the method includes the step of placing an interbody spacer with the vertebrae. In some embodiments, the method includes the step of performing final tightening of bone screws and rotating lock cap(s) to a secured plate position. In some embodiments, the spinal implant system utilizes a method including the step of distracting a disc space to access a posterior and/or posterolateral disc space to perform a step of decompression of a spine.

In some embodiments, the present spinal implant system and method are employed with a surgical technique including a step of decompressing vertebrae. In some embodiments, the method includes the step of resecting the proximal uncovertebral joints along with any osteophytes. In some embodiments, a posterior longitudinal ligament may also be removed, and the nerve roots decompressed. In some embodiments, the method includes the step of inserting an interbody spacer, such as, for example, autograft, allograft or other interbody fusion device in the cervical spine. In some embodiments, the method includes the step of dissecting soft-tissue and removing anterior osteophytes to provide a bone-plate interface.

In some embodiments, the spinal implant system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the spinal implant system of the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The spinal implant system of the present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

In some embodiments, the spinal implant system of the present disclosure includes a plurality of individual plates employed with a surgical technique that includes orienting the individual plates with vertebral tissue in a configuration to maximize a distance between individual plates disposed with adjacent level intervertebral discs. In some embodiments, this orientation of the present individual plates with the vertebral tissue facilitates placement along the disc levels and avoids the complications associated with a single elongated plate that can result in placement to close to an adjacent level disc. As such, this orientation of the present individual plates with the vertebral tissue can avoid adjacent level ossification disease (ALOD) that could lead to adjacent segment degeneration (ASD). In some embodiments, the spinal implant system of the present disclosure includes a plurality of individual plates employed with a surgical technique that includes adding an additional plate, in the same or different surgical procedure, for example, to treat ASD without removal of a previously implanted plate as the present individual plates are oriented with vertebral tissue to provide an amount of vertebral body available for additional plate fixation. In some embodiments the present individual plates are oriented with vertebral tissue to maintain distances with the individual plates to prevent ASD by distancing the individual plates.

In some embodiments, the spinal implant system of the present disclosure includes a plurality of individual plates employed with a surgical technique that includes a four level ACDF. In some embodiments, the four level ACDF with the present individual plates includes two, two vertebral level sites with dual incisions in tissue, which can decrease stress on soft tissues and provides for less stretching of the soft tissues by retractors. As such, the present individual plates employed with the two, two vertebral level sites with dual incisions in tissue provides less postoperative pain and injury potential to soft tissues and esophagus thereby reducing postoperative dysphasia. In some embodiments, the present individual plates employed with dual incisions provides an enhanced view of a spinal construct to optimize ease and precision of screw insertion and plate fixation. In some embodiments, the spinal implant system of the present disclosure includes a plurality of individual plates employed with a surgical technique to facilitate a revision procedure for pseudo-arthrosis. In some embodiments, the present individual plates employed with such revision procedure provides for reduced exposure and dissection of tissue due to the removal of only a single individual plate at a level of pseudo-arthrosis, and the remaining individual plates can remain in place and not disturbed.

In some embodiments, the spinal implant system of the present disclosure includes a plurality of individual plates employed with a surgical technique such that the present individual plates resist and/or prevent overlapping of plate fixation in a cephalad and a caudal direction. In some embodiments, this configuration of the present individual plates provides a reduced stiffness of a global spinal construct while maximizing focal stiffness and compression of an individual spinal level construct. In some embodiments, this configuration of the present individual plates includes 15 mm individual plates oriented with vertebral tissue in a hyper-angulated configuration to resist and/or prevent plate overlap.

The spinal implant system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

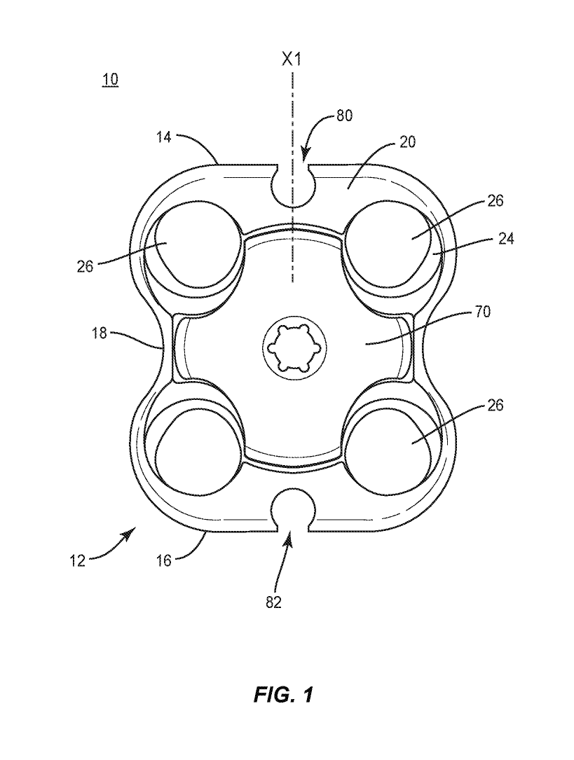

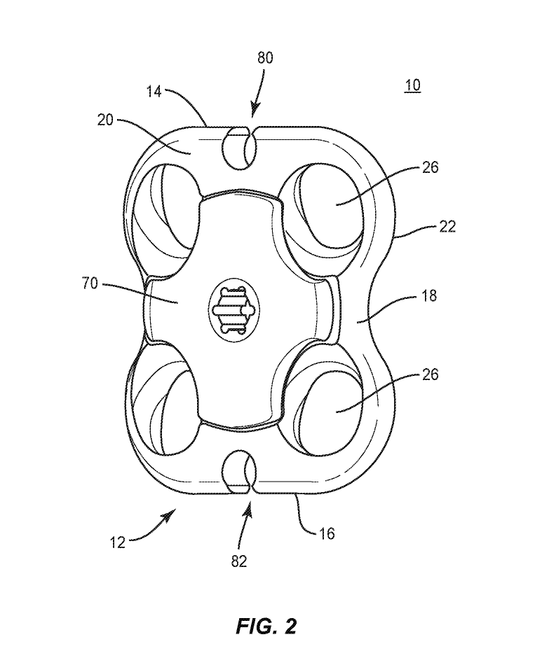

The following discussion includes a description of a spinal implant system, related components and methods of employing the spinal implant system in accordance with the principles of the present disclosure. Alternate embodiments are also disclosed. Reference is made in detail to the exemplary embodiments of the present disclosure, which are illustrated in the accompanying figures. Turning to FIGS. 1 and 2, there are illustrated components of a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 is employed, for example, with an open or mini-open, minimal access and/or minimally invasive including percutaneous surgical technique to deliver and introduce instrumentation and/or spinal implants, such as, for example, cervical plates at a surgical site within a body of a patient, which includes, for example, a single level or multiple levels of vertebrae. Spinal implant system 10 includes a plurality of single level plates employed with a method of treating cervical disorders, for example, multi-level cervical disc degeneration. In some embodiments, each of the multiple single level plates are positioned with vertebrae and physically separated a selected distance from a plate disposed at an adjacent vertebral level. This configuration allows the separated plates to move independently, and provide load sharing, facilitate fusion and avoid imbalance on the individual disc levels. In some embodiments, the multiple single level plates are positioned to allow for multiple fixation screws per level and/or in a hyper-angulated placement to resist pull-out from vertebral tissue. In some embodiments, spinal implant system 10 can include spinal constructs including one or more bone fasteners, interbody implants, cages, spinal rods, tethers and/or connectors, as described herein.

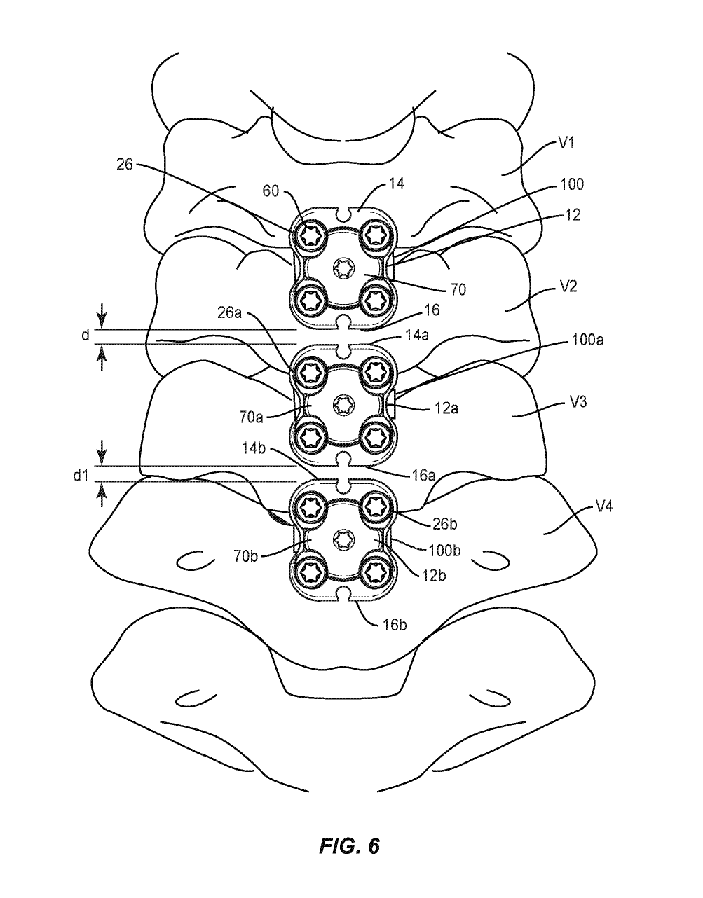

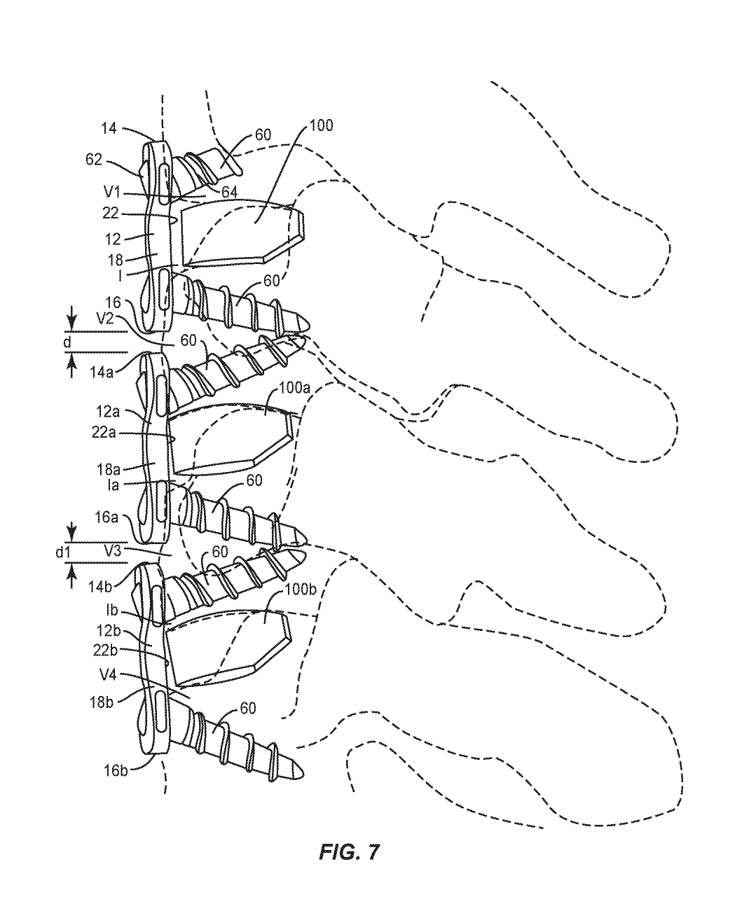

Spinal implant system 10 includes a spinal implant, such as, for example, an anterior cervical plate 12. Plate 12 includes a single level configuration for connecting two vertebral bodies extending along a single vertebral disc space (for example, see FIGS. 6 and 7). In some embodiments, plate 12 has a substantially rectangular shape and a continuous lordotic curve along its length to accommodate the curvature of the spinal column. In some embodiments, plate 12 is variously shaped, such as, for example, oblong, oval, triangular, polygonal, irregular, uniform, non-uniform, variable and/or tapered. In some embodiments, plate 12 includes a length in a range of 15 millimeters (mm) to 21 mm.

Plate 12 extends between a superior most end surface 14 and an inferior most end surface 16. In some embodiments, multiple single level plates 12 are utilized to move with selected vertebrae independently, and provide load sharing, facilitate fusion and avoid imbalance on the individual disc levels. For example, a load applied to vertebrae is transferred to each plate 12 separately to facilitate the independent movement to enhance fusion and maintain balance. In another example, each plate 12 can be disposed adjacent and/or attached with an interbody implant 100 (FIGS. 6 and 7) such that each plate 12 and interbody implant 100 bears a portion of the load applied to the selected vertebrae. In some embodiments, multiple single level plates 12 are disposed in a serial orientation along selected vertebrae. In some embodiments, multiple single level plates 12 having one or more configurations and dimensions are stacked in a selected orientation along selected vertebrae. In some embodiments, multiple single level plates 12 of increasing or decreasing size or dimension are stacked along selected vertebrae. In some embodiments, multiple single level plates 12 of increasing or decreasing size or dimension are stacked in a tapered orientation along selected vertebrae.

Plate 12 includes a wall 18 that extends between surfaces 14, 16. Wall 18 defines a longitudinal axis X1. Wall 18 has a surface 20 configured for orientation in an anterior direction of a body and a surface 22 configured for orientation in a posterior direction to engage an anterior portion of vertebrae. In some embodiments, surface 20 and/or surface 22 may have various surface configurations, such as, for example, rough, threaded, arcuate, undulating, porous, semi-porous, dimpled, polished and/or textured.

Wall 18 includes a surface 24 that defines openings 26 configured for disposal of a bone screw 60, as described herein. Each opening 26 extends between surfaces 20, 22. Openings 26 are substantially circular and extend through the thickness of wall 18. In some embodiments, openings 26 may be disposed at alternate orientations, relative to wall 18 and/or axis X1, such as, for example, substantially transverse, perpendicular, parallel and/or other angular orientations such as acute or obtuse, and/or may be offset. In some embodiments, openings 26 can be variously configured, such as, for example, oval, oblong, triangular, square, polygonal, irregular, uniform, non-uniform, tapered and/or countersunk. In some embodiments, plate 12 can have one or a plurality of openings 26.

Bone screw 60 comprises a head 62 and an elongated shaft 64 configured for penetrating tissue. In some embodiments, spinal implant system 10 may include one or a plurality of bone fasteners and/or penetrating elements. Shaft 64 has a cylindrical cross section configuration and includes an outer surface having an external threaded form. In some embodiments, the thread form may include a single thread turn or a plurality of discrete threads. In some embodiments, other engaging structures may be located on shaft 64, such as, for example, a nail configuration, barbs, expanding elements, raised elements and/or spikes to facilitate engagement of shaft 64 with tissue, such as, for example, vertebrae.

In some embodiments, all or only a portion of shaft 64 may have alternate cross section configurations, such as, for example, oval, oblong, triangular, square, polygonal, irregular, uniform, non-uniform, offset, staggered, undulating, arcuate, variable and/or tapered. In some embodiments, shaft 64 may include one or a plurality of openings. In some embodiments, all or only a portion of shaft 64 may have alternate surface configurations to enhance fixation with tissue such as, for example, rough, arcuate, undulating, mesh, porous, semi-porous, dimpled and/or textured. In some embodiments, all or only a portion of shaft 64 may be disposed at alternate orientations, relative to its longitudinal axis, such as, for example, transverse, perpendicular and/or other angular orientations such as acute or obtuse, co-axial and/or may be offset or staggered. In some embodiments, all or only a portion of bone screw 60 may be cannulated.

In some embodiments, plate 12 includes a retaining element 70 disposed between openings 26 to resist and/or prevent inadvertent back out of bone screws 60 after bone screws 60 have been fully inserted into openings 26. In some embodiments, retaining element 70 is configured to move between a locked orientation and a non-locking orientation. In the locked orientation, retaining element 70 resists and/or prevents back out of bone screws 60 from openings 26. In the non-locking orientation, bone screws 60 are axially translatable through openings 26.

In some embodiments, end 14 includes a surface that defines a cavity 80 configured for disposal and capture of a distraction pin 80a (FIG. 4). In some embodiments, end 16 includes a surface that defines a cavity 82 configured for disposal and capture of a distraction pin 80b (FIG. 4).

In assembly, operation and use, spinal implant system 10, similar to the systems and methods described herein, is employed with a surgical procedure for treatment of a spinal disorder affecting a section of a spine of a patient, as discussed herein. For example, spinal implant system 10 can be used with a surgical procedure for treatment of a condition or injury of an affected section of the spine including vertebrae. In some embodiments, one or all of the components of spinal implant system 10 can be delivered or implanted as a pre-assembled device or can be assembled in situ. Spinal implant system 10 may be completely or partially revised, removed or replaced.

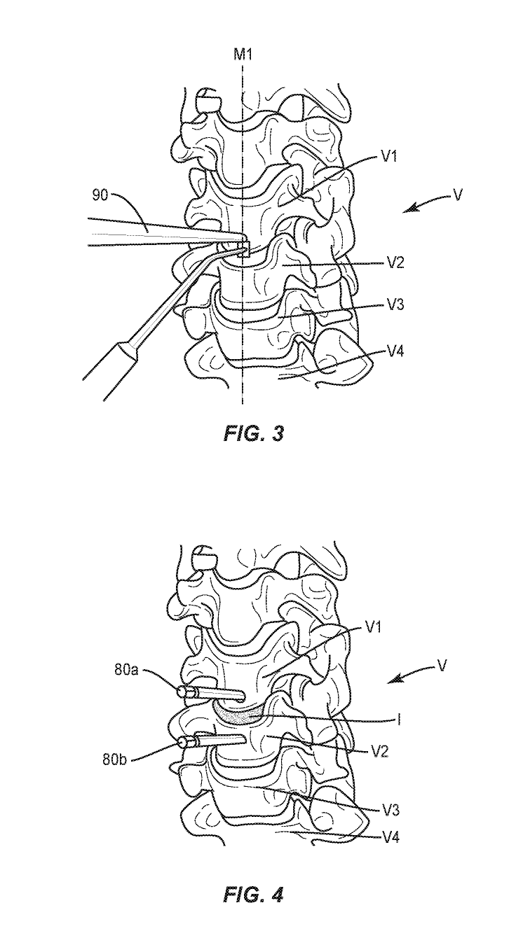

For example, spinal implant system 10 can be employed with a surgical treatment of an applicable condition or injury of an affected section of a spinal column and adjacent areas within a body, such as, for example, cervical vertebrae V, as shown in FIGS. 3-7. In some embodiments, spinal implant system 10 may be employed with one or a plurality of vertebra. To treat a selected section of vertebrae V, a medical practitioner obtains access to a surgical site including vertebrae V in any appropriate manner. In some embodiments, spinal implant system 10 can be used in any existing surgical method or technique including open surgery, mini-open surgery, minimally invasive surgery and percutaneous surgical implantation, whereby vertebrae V are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for implantation of components of spinal implant system 10. Tissue is spaced with a retractor (not shown). A preparation instrument (not shown) can be employed to prepare tissue surfaces of vertebrae V, as well as for aspiration and irrigation of a surgical region. In some embodiments, the surgical procedure includes making dual incisions in the body of the patient to create two vertebral level sites for placement of individual plates 12, as described herein, which can decrease stress and stretching of the soft tissues by retractors, resulting in less postoperative pain and injury potential. In some embodiments, the surgical procedure including dual incisions provides an enhanced view of a spinal construct, as described herein, to optimize ease and precision of screw insertion and plate fixation.

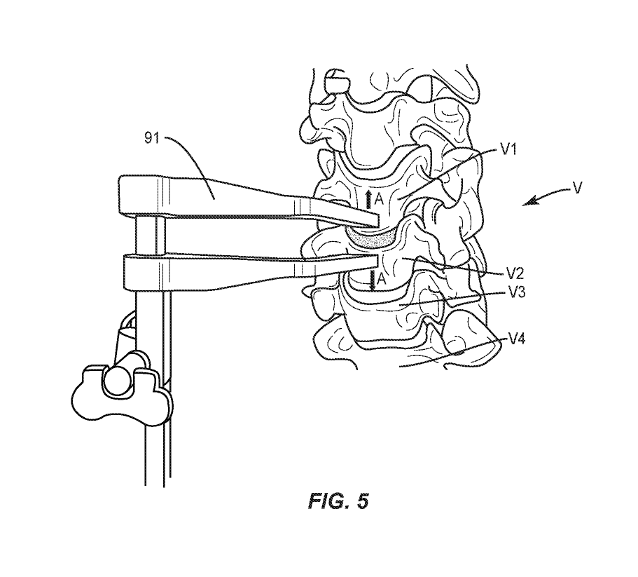

Vertebrae V1, V2 are prepared for attachment of plate 12. For example, a distraction pin placement tool 90, as shown in FIG. 3, is employed to insert a distraction pin 80a in vertebral body V1 and a second distraction pin 80b in vertebral body V2 parallel to distraction pin 80a, as shown in FIG. 4. In some embodiments, a distraction tool 91, as shown in FIG. 5, is employed to distract, in the directions shown by arrows A, an intervertebral disc space I to access the posterior and/or posterolateral disc space for decompression of the spine.

In some embodiments, a discectomy is performed. In some embodiments, the proximal uncovertebral joints may be resected along with any osteophytes. In some embodiments, the posterior longitudinal ligament may be removed, and the nerve roots decompressed. In some embodiments, an interbody implant 100 is implanted with space I, as shown in FIGS. 6 and 7. In some embodiments, interbody implant 100 may include, such as, for example, autograft, allograft or any interbody fusion device.

Plate 12 is delivered to the surgical site adjacent vertebrae V1, V2 with an insertion tool (not shown) and over distraction pins 80a, 80b. Plate 12 is disposed in a selected position and orientation relative to vertebrae V1, V2. Plate 12 extends along intervertebral disc space I such that surface 22 of plate 12 is disposed to engage anterior tissue surfaces of vertebrae V1, V2 with disc space I disposed therebetween.

Bone screws 60 are delivered to the surgical site and disposed in openings 26 and engaged with vertebrae V1, V2. The components of spinal implant system 10 include a driver (not shown) that is manipulable to drive, torque, insert or otherwise connect bone screws 60 with vertebrae V1, V2 for fastening plate 12 with vertebrae V1, V2. In some embodiments, bone screws 60 are oriented hyper angulated relative to wall 18 and/or axis X1 of plate 12 to resist pull-out from vertebrae V1, V2, as shown in FIG. 7. In some embodiments, bone screws 60 are angled in a range of 0 to 20 degrees relative to wall 18 and/or axis X1. A driver (not shown) is positioned within retaining element 70 to rotate retaining element 70 from the non-locking orientation to the locked orientation, such that retaining element 70 partially overlaps bone screws 60 and openings 26 to resist and/or prevent inadvertent back out of bone screws 60 from plate 12 and/or tissue.

In some embodiments, a discectomy is performed and vertebrae V2, V3 are distracted, similar to that described with regard to vertebrae V1, V2. An interbody implant 100a is disposed with intervertebral disc space Ia. A plate 12a is delivered to the surgical site adjacent vertebrae V2, V3 with the insertion tool. Plate 12a is disposed in a selected position and orientation relative to vertebrae V2, V3. Plate 12a extends along intervertebral disc space Ia such that a surface 22a of plate 12a is disposed to engage anterior tissue surfaces of vertebrae V2, V3 with disc space Ia disposed therebetween.

Plate 12 is disposed with vertebrae V1, V2 and plate 12a is disposed with vertebrae V2, V3 such that plates 12, 12a are disposed in alignment along vertebrae V and axially spaced apart a selected distance. In some embodiments, the selected distance includes a gap d between plates 12, 12a in a cranial-caudal orientation relative to vertebrae V. In some embodiments, plates 12, 12a are spaced apart by a gap d of at least 1 mm. As such, inferior most end surface 16 of plate 12 is disposed axially superior the selected distance relative to a superior most end surface 14a of plate 12a. Plates 12, 12a are positioned with vertebrae V and physically separated the selected distance. This configuration allows separated plates 12, 12a to move independently, and provide load sharing, facilitate fusion and avoid imbalance on individual disc levels I, Ia. A load applied to vertebrae V is transferred to each plate 12, 12a and/or interbody implants 100, 100a separately to facilitate the independent movement to enhance fusion and maintain balance.

Bone screws 60 are delivered to the surgical site and disposed in openings 26a and engaged with vertebrae V2, V3. The components of spinal implant system 10 include a driver (not shown) that is manipulable to drive, torque, insert or otherwise connect bone screws 60 with vertebrae V2, V3 for fastening plate 12a with vertebrae V2, V3. In some embodiments, bone screws 60 are oriented hyper angulated relative to a wall 18a and/or an axis of plate 12a to resist pull-out from vertebrae V2, V3, as shown in FIG. 7. In some embodiments, bone screws 60 are angled in a range of 0 to 20 degrees relative to wall 18a and/or the axis of plate 12a. A driver (not shown) is positioned within retaining element 70a to rotate retaining element 70a from the non-locking orientation to the locked orientation, such that retaining element 70a partially overlaps bone screws 60 and openings 26a to resist and/or prevent inadvertent back out of bone screws 60 from plate 12a and/or tissue.

In some embodiments, a discectomy is performed and vertebrae V3, V4 are distracted, similar to that described with regard to vertebrae V1, V2, V3. An interbody implant 100b is disposed with intervertebral disc space Ib. A plate 12b is delivered to the surgical site adjacent vertebrae V3, V4 with the insertion tool. Plate 12b is disposed in a selected position and orientation relative to vertebrae V3, V4. Plate 12b extends along intervertebral disc space Ib such that a surface 22b of plate 12b is disposed to engage anterior tissue surfaces of vertebrae V3, V4 with disc space Ib disposed therebetween.

Plate 12a is disposed with vertebrae V2, V3 and plate 12b is disposed with vertebrae V3, V4 such that plates 12a, 12b are disposed in alignment along vertebrae V and axially spaced apart a selected distance. In some embodiments, the selected distance includes a gap d1 between plates 12a, 12b in a cranial-caudal orientation relative to vertebrae V. In some embodiments, plates 12a, 12b are spaced apart by a gap d1 of at least 1 mm. As such, inferior most end surface 16a of plate 12a is disposed axially superior the selected distance relative to a superior most end surface 14b of plate 12b. Plates 12a, 12b are positioned with vertebrae V and physically separated the selected distance. This configuration allows separated plates 12, 12a, 12b to move independently, and provide load sharing, facilitate fusion and avoid imbalance on individual disc levels I, Ia, Ib. A load applied to vertebrae V is transferred to each plate 12, 12a, 12b and/or interbody implants 100, 100a, 100b separately to facilitate the independent movement to enhance fusion and maintain balance. In some embodiments, a selected distance, as described herein and for example the selected distance between plates 12, 12a and/or plates 12a, 12b, can be selected in a range of 1 through 75 mm. In some embodiments, the selected distance includes 5 mm. In some embodiments, the selected distance includes 10 mm. In some embodiments, the selected distance includes 25 mm. In some embodiments, the selected distance between plates 12, 12a and plates 12a, 12b can be different, equal, increasing or decreasing.

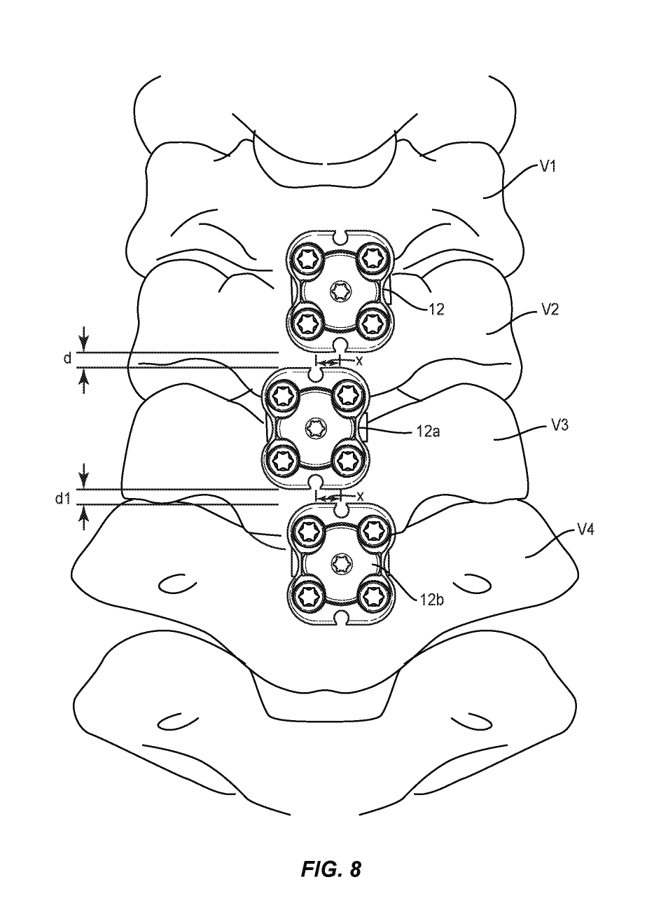

Bone screws 60 are delivered to the surgical site and disposed in openings 26b and engaged with vertebrae V3, V4. The components of spinal implant system 10 include a driver (not shown) that is manipulable to drive, torque, insert or otherwise connect bone screws 60 with vertebrae V3, V4 for fastening plate 12b with vertebrae V3, V4. In some embodiments, bone screws 60 are oriented hyper angulated relative to a wall 18b and/or an axis of plate 12b to resist pull-out from vertebrae V3, V4, as shown in FIG. 7. In some embodiments, bone screws 60 are angled in a range of 0 to 20 degrees relative to wall 18b and/or the axis of plate 12b. A driver (not shown) is positioned within retaining element 70b to rotate retaining element 70b from the non-locking orientation to the locked orientation, such that retaining element 70b partially overlaps bone screws 60 and openings 26b to resist and/or prevent inadvertent back out of bone screws 60 from plate 12b and/or tissue. In some embodiments, spinal implant system 10 can include one or more cervical plates disposed in alignment along vertebrae V and axially spaced apart selected distances, as described herein. In some embodiments, spinal implant system 10, as described herein, includes plates 12, 12a, 12b disposed relatively offset laterally a distance x and/or alternating along vertebrae V, and spaced apart by gaps d, d1, respectively, as shown in FIG. 8. In some embodiments, distance x includes a distance in a range of 1-5 mm. The offset orientation of plates 12, 12a, 12b facilitates screw placement and/or avoids interference between adjacent screws 60.

Upon completion of the procedure, the surgical instruments, assemblies and non-implanted components of spinal implant system 10 are removed from the surgical site and the incision is closed. One or more of the components of spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components of spinal implant system 10 with vertebral tissue. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating vertebrae, the method comprising the steps of:
attaching a first spinal plate with cervical vertebrae to extend along a first intervertebral disc space; and
attaching a second spinal plate with cervical vertebrae to extend along a second intervertebral disc space such that the plates are axially spaced apart a selected distance.

2. A method as recited in claim 1, wherein the plates are disposed in a serial alignment along an axis of the cervical vertebrae.

3. A method as recited in claim 1, wherein the plates are disposed in an offset configuration along an axis of the cervical vertebrae.

4. A method as recited in claim 1, wherein the first plate includes an inferior most end surface and the second plate includes a superior most end surface, the inferior most end surface being disposed axially superior the selected distance relative to the superior most end surface.

5. A method as recited in claim 1, further comprising the step of attaching a third spinal plate with cervical vertebrae to extend along a third intervertebral disc space such that the third plate and the second plate are disposed in alignment and axially spaced apart a selected distance.

6. A method as recited in claim 5, wherein the second plate includes an inferior most end surface and the third plate includes a superior most end surface, the inferior most end surface being disposed axially superior the selected distance relative to the superior most end surface.

7. A method as recited in claim 5, wherein the plates are disposed in a serial alignment along an axis of the cervical vertebrae.

8. A method as recited in claim 1, wherein the first plate includes at least one opening for disposal of a bone screw that attaches the first plate with the cervical vertebrae.

9. A method as recited in claim 8, wherein the bone screw is fixed with the cervical vertebrae and disposed at an angular orientation relative to the first plate.

10. A method as recited in claim 1, wherein the method comprises a multiple vertebral level ACDF.

11. A method as recited in claim 1, wherein the cervical vertebrae includes at least a first vertebral body and a second vertebral body, and further comprising the step of distracting the first vertebral body relative to the second vertebral body.

12. A method as recited in claim 11, further comprising the step of implanting an interbody implant between the first vertebral body and the second vertebral body.

13. A method as recited in claim 1, further comprising the step of performing a discectomy and/or a decompression.

14. A method for treating vertebrae, the method comprising the steps of:
attaching a plurality of spinal plates in a serial configuration axially along cervical vertebrae, the spinal plates being spaced apart a selected distance and are independently movable relative to each other; and
implanting an interbody implant between a first vertebral body and a second vertebral body of the cervical vertebrae.

15. A method as recited in claim 14, wherein each of the plurality of spinal plates includes at least one opening for disposal of a bone screw disposed at an angular orientation relative to the plate.

16. A method as recited in claim 15, wherein the bone screws are hyper-angulated relative to the plate.

17. A method as recited in claim 14, wherein each of the plurality of plates includes an inferior most end surface and an adjacent plate includes a superior most end surface, the inferior most end surface being disposed axially superior the selected distance relative to the superior most end surface.

18. A method as recited in claim 17, wherein the step of attaching the plurality of spinal plates includes attaching a first plate along a first intervertebral disc;
attaching a second plate along a second intervertebral disc a selected distance from the first plate; and
attaching a third plate along a third intervertebral disc space a selected distance from the second plate.

19. A method as recited in claim 14, wherein the method comprises a multiple vertebral level ACDF.

20. A method for treating vertebrae, the method comprising the steps of:
disposing a first spinal plate with cervical vertebrae to extend along a first intervertebral disc space between a first vertebral body and a second vertebral body;
attaching bone screws with the vertebral bodies via openings of the first plate such that the bone screws are hyper-angulated relative to the first plate;
disposing a second spinal plate with cervical vertebrae to extend along a second intervertebral disc space between the second vertebral body and a third vertebral body such that the plates are disposed in alignment and axially spaced apart a selected distance; and
attaching bone screws with the second vertebral body and the third vertebral body via openings of the second plate such that the bone screws are hyper-angulated relative to the second plate.
